(19) United States
(12) Patent Application Publication
(10) Pub. No.: US 2010/0261244 A1
(43) Pub. Date: Oct. 14, 2010

(54) METHOD FOR IMMOBILIZING BIO-MATERIAL ON TITANIUM DIOXIDE NANOPARTICLES AND TITANIUM DIOXIDE NANOPARTICLES IMMOBILIZED BY BIO-MATERIAL

(75) Inventors: Wan Joong Kim, Gyunggi-do (KR); Gun Yong Sung, Daejeon (KR); Seon Hee Park, Daejeon (KR); Hyun Sung Ko, Daejeon (KR); Chul Huh, Daejeon (KR); Kyung Hyun Kim, Daejeon (KR); Jong Cheol Hong, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/743,340

(22) PCT Filed: Jun. 5, 2008
(86) PCT No.: PCT/KR08/03179

§ 371 Date: May 17, 2010

(30) Foreign Application Priority Data
Dec. 17, 2007 (KR) 10-2007-0132292

(51) Int. Cl. C12N 11/02; C07F 7/28; C07H 21/02; C07H 21/04; C07K 14/00

(52) U.S. Cl. 435/177; 556/51; 536/23.1; 530/400; 977/773

(57) ABSTRACT

There is provided a method for immobilizing a bio-material on a surface of titanium dioxide nanoparticles (TiO₂) as a highly reflective material to enhance sensitivity of a resonant reflection biosensor. The method for immobilizing a bio-material may be useful to easily immobilize bio-materials such as proteins, DNA, RNA and enzymes on surfaces of titanium dioxide (TiO₂) nanoparticles using the chemical reaction, and significantly improve sensitivity of a resonant reflection biosensor by determining the antigen-antibody reaction in the resonant reflection biosensor using the immobilized secondary antien.

### TECHNICAL FIELD

The present invention relates to a method for immobilizing a bio-material on a surface of titanium dioxide nanoparticles (TiO₂) as a highly reflective material to enhance sensitivity of a resonant reflection biosensor, and more particularly, to a method for immobilizing a bio-material on titanium dioxide nanoparticles (TiO₂) nanoparticles using the surface reaction of a bio-material such as protein, DNA, RNA, enzyme, etc.

The present invention was supported by the Information Technology Research and Development (IT R&D) Program of Ministry of Information and Communication (MIC) [2006-S-007-02, Immobilization of protein, DNA, RNA and Enzyme on TiO₂ nanoparticles)].

### BACKGROUND ART

Resonant reflection biosensors have been used to determine the presence of the antigen-antibody reaction by measuring only the changes in optical thickness, contrary to determining the presence of the antigen-antibody reaction through labeling with fluorescent substances, isotopes and pigments in the conventional immunoassays. That is to say, the sensitivity of the resonant reflection biosensor are determined by the changes in the optical thickness before/after the antigen-antibody reaction.

However, the antigens generally have a size of about 5 to 10 nm, and the sensitivity of the resonant reflection biosensor is restricted again according to the density of surface-immobilized antibody. Therefore, the problem is that it is difficult to measure the changes in the optical thickness accurately.

Therefore, there is an increasing demand for a method capable of increasing the changes in optical thickness to confirm the presence of the antigen-antibody reaction using a resonant reflection biosensor.

### DISCLOSURE OF INVENTION

### Technical Problem

The present invention is designed to solve the problems of the prior art, and therefore it is an object of the present invention to provide a method for immobilizing a bio-material on titanium dioxide nanoparticles (TiO₂) nanoparticles using the surface reaction of a bio-material such as protein, DNA, RNA, enzyme, etc.

Also, it is another object of the present invention to provide titanium dioxide nanoparticles (TiO₂) capable of enhancing sensitivity of a resonant reflection biosensor

### Technical Solution

According to an aspect of the present invention, there is provided a titanium dioxide (TiO₂) nanoparticle immobilized by a bio-material, including titanium dioxide (TiO₂) having a hydroxyl (—OH) group formed in a surface thereof; an aldehyde (—CHO) group layer engrafted into the hydroxyl (—OH) group of titanium dioxide (TiO₂) using a self-assembly method; and a bio-material immobilized on the aldehyde (—CHO) group layer.

In this case, the titanium dioxide (TiO₂) may have the hydroxyl (—OH) group formed through the reaction with a piranha solution, and the aldehyde (—CHO) group layer may be formed through the reaction of an aldehyde silane solution with the titanium dioxide (TiO₂) having a hydroxyl (—OH) group formed in the surface thereof.

In addition, the bio-material may be selected from the group consisting of proteins, DNA, RNA and enzymes.

According to another aspect of the present invention, there is provided a method for immobilizing a bio-material on titanium dioxide (TiO₂) nanoparticles, the method including: binding hydroxyl (—OH) group to titanium dioxide (TiO₂) nanoparticles through the reaction with a piranha solution; forming aldehyde (—CHO) group on the hydroxyl (—OH)-bound titanium dioxide (TiO₂) nanoparticles through the reaction with an aldehyde silane solution; and immobilizing a bio-material on the titanium dioxide (TiO₂) nanoparticles through the reaction with the aldehyde group (—CHO) of the titanium dioxide (TiO₂).

In this case, the binding of a hydroxyl (—OH) group may include: heating the titanium dioxide (TiO₂) nanoparticles in a piranha solution; and separating the hydroxyl (—OH)-bound titanium dioxide (TiO₂) nanoparticles by using a centrifuge after the heating operation. Also, the separating of the titanium dioxide (TiO₂) nanoparticles may further include: washing the titanium dioxide (TiO₂) nanoparticles with double-distilled water.

Furthermore, the forming of an aldehyde (—CHO) group may include: heating the hydroxyl (—OH)-bound titanium dioxide (TiO₂) nanoparticles in an aldehyde silane solution; and separating the hydroxyl (—OH)-bound titanium dioxide (TiO₂) nanoparticles having aldehyde (—CHO) group formed in external surfaces thereof from the aldehyde silane solution by using a centrifuge.

According to still another aspect of the present invention, there is provided a method for immobilizing a bio-material on titanium dioxide (TiO₂) nanoparticles, the method including: binding hydroxyl (—OH) group to titanium dioxide (TiO₂) nanoparticles through the reaction with a piranha solution; forming amino (—NH₂) group on the hydroxyl (—OH)-bound titanium dioxide (TiO₂) nanoparticles through the reaction with an aminosilane (3-aminopropyltriethoxysilane) solution; forming aldehyde (—CHO) group on the amino group (—NH₂)-grafted titanium dioxide (TiO₂) nanoparticles through the reaction with glutaraldehyde; and immobilizing a bio-material on the titanium dioxide (TiO₂) nanoparticles through the reaction with the aldehyde group (—CHO) of the titanium dioxide (TiO₂).

According to yet another aspect of the present invention, there is provided a method for immobilizing a bio-material on titanium dioxide (TiO₂) nanoparticles, the method including: binding hydroxyl (—OH) group to titanium dioxide (TiO₂) nanoparticles through the reaction with a piranha solution; forming amino (—NH₂) group on the hydroxyl (—OH)-bound titanium dioxide (TiO₂) nanoparticles through the reaction with an aminosilane (3-aminopropyltriethoxysilane) solution; engrafting maleimido group into the amino group (—NH₂)-grafted titanium dioxide (TiO₂) nanoparticles using succinimidyl-4-(p-maleimide phenyl)butyrate (SMPB) as a cross-linker; engrafting 3Cys-protein G into the maleimido group-grafted titanium dioxide (TiO₂) nanoparticles when the maleimido group is engrafted into the titanium dioxide (TiO₂) nanoparticles; and immobilizing a bio-material on the titanium dioxide (TiO₂) nanoparticles through the reaction with the 3Cys-protein G of the titanium dioxide (TiO₂) nanoparticles.

### Advantageous Effects

As described above, the titanium dioxide (TiO₂) nanoparticles immobilized by a bio-material according to the present invention, and the method for immobilizing a bio-material on titanium dioxide (TiO₂) nanoparticles may be useful to significantly improve sensitivity of a resonant reflection biosensor by increasing the changes in optical thickness that appear in the antibody-antigen reaction using the resonant reflection biosensor.

### BRIEF DESCRIPTION OF THE DRAWINGS

FIG. 1 is a diagram illustrating a method for immobilizing a bio-material on titanium dioxide (TiO₂) nanoparticles according to one exemplary embodiment of the present invention.

FIG. 2 is a diagram illustrating an FE-SEM photograph in which the titanium dioxide (TiO₂) nanoparticles used in the present invention are magnified 100,000 times (500 nm×200 nm in size).

FIG. 3 is a diagram illustrating the infrared ray (IR) spectroscopic results of an aldehyde group (—CHO) materials engrafted into the titanium dioxide (TiO₂) nanoparticles according to one exemplary embodiment of the present invention.

FIG. 4 is a diagram illustrating the titanium dioxide (TiO₂) nanoparticles immobilized by a bio-material according to one exemplary embodiment of the present invention.

FIG. 5 is a schematic view illustrating an operation of measuring titanium dioxide (TiO₂) nanoparticles in a resonant reflection biosensor using antibody labeled with the titanium dioxide (TiO₂) nanoparticles according to one exemplary embodiment of the present invention.

FIG. 6 is a diagram illustrating the spectrum results of the titanium dioxide (TiO₂) nanoparticles, which are immobilized by the bio-material, in a resonant reflection biosensor according to one exemplary embodiment of the present invention.

FIG. 7 is a diagram illustrating the spectrum results of the titanium dioxide (TiO₂) nanoparticles in the resonant reflection biosensor when a bio-material is not immobilized on the titanium dioxide (TiO₂) nanoparticles.

### BEST MODE FOR CARRYING OUT THE INVENTION

Hereinafter, exemplary embodiments of the present invention will now be described in detail with reference to the accompanying drawings, for the purpose of better understanding of the present invention as apparent to those skilled in the art. For the detailed description of the present invention, it is however considered that descriptions of known functions and their related configurations according to the exemplary embodiments of the present invention may be omitted when they are judged to make the gist of the present invention unclear.

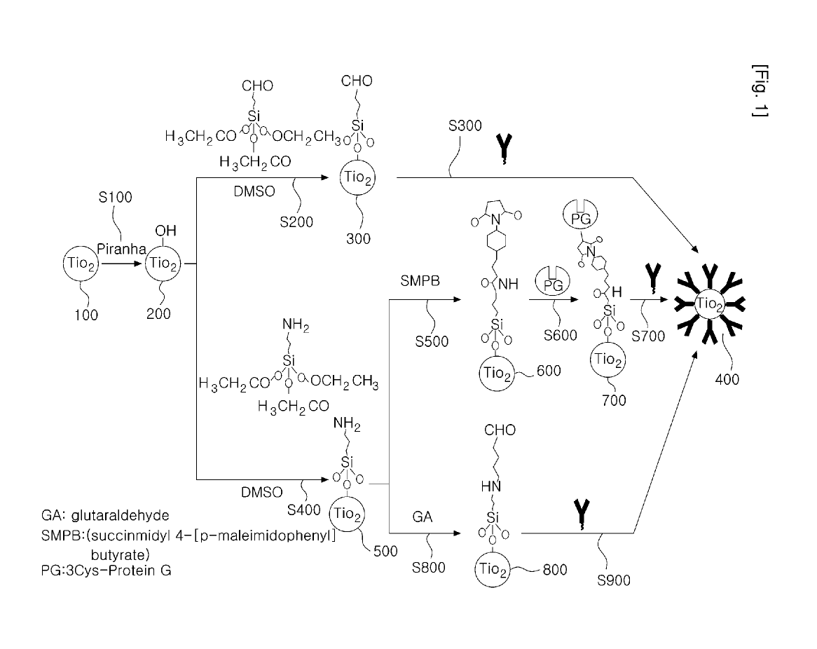

FIG. 1 is a diagram illustrating a method for immobilizing a bio-material on titanium dioxide (TiO₂) nanoparticles according to one exemplary embodiment of the present invention.

The method for immobilizing a bio-material on titanium dioxide (TiO₂) nanoparticles includes: binding hydroxyl (—OH) group to titanium dioxide (TiO₂) nanoparticles 100 through the reaction with a piranha solution (S100); forming aldehyde (—CHO) group on the hydroxyl (—OH)-bound titanium dioxide (TiO₂) nanoparticles 200 through the reaction with an aldehyde silane solution (S200); and immobilizing a bio-material on the titanium dioxide (TiO₂) nanoparticles through the reaction with the aldehyde group (—CHO) of the titanium dioxide (TiO₂) (S300).

First, hydroxyl (—OH) group is engrafted into the titanium dioxide (TiO₂) nanoparticles 100 (S100). As a result, the titanium dioxide (TiO₂) nanoparticles 200 grafted with the hydroxyl (—OH) group that forms a self-assembled monolayer are obtained. More particularly, the titanium dioxide (TiO₂) nanoparticles 100 with a diameter of about 30 nm are added to a piranha solution (sulfuric acid:30% hydrogen peroxide=3:1). Then, the piranha solution is heated at about 80? for at least one hour. After the heating of the piranha solution, the piranha solution is washed several times with double-distilled water using a centrifuge to obtain hydroxyl (—OH)-bound titanium dioxide (TiO₂) nanoparticles 200.

Next, aldehyde (—CHO) group is formed in the hydroxyl (—OH)-bound titanium dioxide (TiO₂) nanoparticles 200 (S200). Here, the binding of aldehyde group (—CHO) is carried out through the self-assembly so as to bind a bio-material such as protein to the hydroxyl (—OH)-bound titanium dioxide (TiO₂) nanoparticles. More particularly, the hydroxyl (—OH)-bound titanium dioxide (TiO₂) nanoparticles 200 are put into an aldehyde silane solution, and the resulting mixture is heated at about 85? for 24 hours. The heated mixture is washed with dimethyl sulfoxide (DMSO) using a centrifuge. And, the aldehyde silane solution is changed with a PBS buffer.

When the aldehyde group (—CHO)-bound titanium dioxide (TiO₂) nanoparticles 300 are formed in the PBS buffer, a bio-material such as protein, DNA, RNA and enzyme is put into the PBS buffer solution, and the resulting mixture is stirred at a room temperature for about 12 hours. In this reaction, the bio-material such as a protein, DNA, RNA and an enzyme is immobilized on the titanium dioxide (TiO₂) nanoparticles.

As an alternative, the method for immobilizing a bio-material on titanium dioxide (TiO₂) nanoparticles includes: binding hydroxyl (—OH) group to titanium dioxide (TiO₂) nanoparticles 100 through the reaction with a piranha solution (S100); forming amino (—NH₂) group on the hydroxyl (—OH)-bound titanium dioxide (TiO₂) nanoparticles through the reaction with an aminosilane (3-aminopropyltriethoxysilane) solution (S400); forming aldehyde (—CHO) group on the amino group (—NH₂)-grafted titanium dioxide (TiO₂) nanoparticles using glutaraldehyde (S800); and immobilizing a bio-material on the titanium dioxide (TiO₂) nanoparticles (S900).

First, hydroxyl (—OH) group is engrafted into the titanium dioxide (TiO₂) nanoparticles 100 (S100). As a result, the titanium dioxide (TiO₂) nanoparticles 200 grafted with the hydroxyl (—OH) group that forms a self-assembled monolayer are obtained. More particularly, the titanium dioxide (TiO₂) nanoparticles 100 with a diameter of about 30 nm are added to a piranha solution (sulfuric acid:30% hydrogen peroxide=3:1). Then, the piranha solution is heated at about 80? for at least one hour. After the heating of the piranha solution, the piranha solution is washed several times with double-distilled water using a centrifuge to obtain hydroxyl (—OH)-bound titanium dioxide (TiO₂) nanoparticles 200.

Next, the hydroxyl (—OH)-bound titanium dioxide (TiO₂) nanoparticles 200 are added to a 0.1% aminosilane (3-aminopropyltriethoxysilane) solution, and the resulting mixture is heated at about 85? for 24 hours. The heated mixture is washed with dimethyl sulfoxide (DMSO) using a centrifuge. And, the aldehyde silane solution is changed with a PBS buffer.

25% glutaraldehyde is added to the amino group (—NH₂)-bound titanium dioxide (TiO₂) nanoparticles 500, and stirred for 12 hours. Then, the resulting mixture is washed with distilled water using a centrifuge. After aldehyde (—CHO) group is engrafted into the titanium dioxide (TiO₂) nanoparticles 500 (S800), an operation of immobilizing a bio-material on titanium dioxide (TiO₂) nanoparticles is carried out (S900). A bio-material such as a protein, DNA, RNA and an enzyme is added to a PBS buffer, and stirred at a room temperature for 12 hours. In this reaction, the bio-material such as a protein, DNA, RNA and an enzyme is immobilized on the titanium dioxide (TiO₂) nanoparticles.

As another alternative, the method for immobilizing a bio-material on titanium dioxide (TiO₂) nanoparticles capable of improving orientation of the titanium dioxide (TiO₂) nanoparticles includes: binding hydroxyl (—OH) group to titanium dioxide (TiO₂) nanoparticles 100 through the reaction with a piranha solution (S100); forming amino (—NH₂) group on the hydroxyl (—OH)-bound titanium dioxide (TiO₂) nanoparticles 200 through the reaction with an aminosilane (3-aminopropyltriethoxysilane) solution (S400); engrafting maleimido group into the amino group (—NH₂)-grafted titanium dioxide (TiO₂) nanoparticles using succinimidyl-4-(p-maleimide phenyl)butyrate (SMPB) as a cross-linker to bind the amino group (—NH₂)-grafted titanium dioxide (TiO₂) nanoparticles to protein G (S500); engrafting 3Cys-protein G into the maleimido group-grafted titanium dioxide (TiO₂) nanoparticles (S600); and immobilizing a bio-material on the titanium dioxide (TiO₂) nanoparticles through the reaction with the 3Cys-protein G of the titanium dioxide (TiO₂) nanoparticles (S700).

More particularly, hydroxyl (—OH) group is engrafted into the titanium dioxide (TiO₂) nanoparticles 100 (S100). As a result, the titanium dioxide (TiO₂) nanoparticles 200 grafted with the hydroxyl (—OH) group that forms a self-assembled monolayer are obtained.

Next, the hydroxyl (—OH)-bound titanium dioxide (TiO₂) nanoparticles 200 are added to a 0.1% aminosilane (3-aminopropyltriethoxysilane) solution, and the resulting mixture is heated at about 85? for 24 hours. The heated mixture is washed with dimethyl sulfoxide (DMSO) using a centrifuge.

4 μmol SMPB is added to the amino group (—NH₂)-bound titanium dioxide (TiO₂) nanoparticles 400 and stirred for 6 hours. Then, the resulting mixture is washed with dimethyl sulfoxide (DMSO) using a centrifuge, and the aldehyde silane solution is changed with a PBS buffer. 1 mol 3Cys-protein G is added to the maleimido group-grafted titanium dioxide (TiO₂) nanoparticles 600 and stirred for 12 hours. Then, the resulting mixture is washed with PBS buffer using a centrifuge. After the protein G is engrafted into the titanium dioxide (TiO₂) nanoparticles 600 (7800), an operation of immobilizing a bio-material on titanium dioxide (TiO₂) nanoparticles is carried out (S900). A bio-material such as a protein, DNA, RNA and an enzyme is added to a PBS buffer, and stirred at a room temperature for 12 hours. In this reaction, the bio-material such as a protein, DNA, RNA and an enzyme is immobilized on the titanium dioxide (TiO₂) nanoparticles.

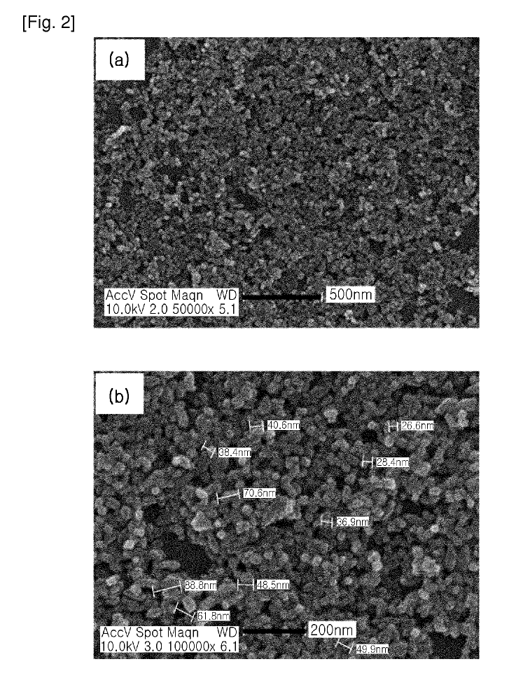

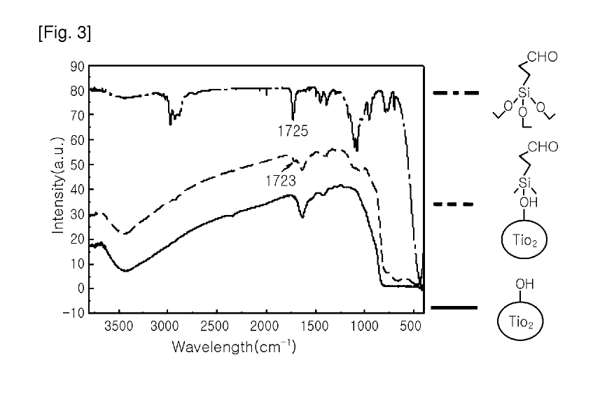

FIG. 2 is a diagram illustrating an FE-SEM photograph in which the titanium dioxide (TiO₂) nanoparticles used in the present invention are magnified 100,000 times (500 nm×200 nm in size), and FIG. 3 is a diagram illustrating the infrared ray (IR) spectroscopic results of an aldehyde group (—CHO) materials engrafted into the titanium dioxide (TiO₂) nanoparticles according to the present invention.

As shown in FIG. 2, the titanium dioxide (TiO₂) nanoparticles have a size of about 30 nm according to the FE-SEM photograph of the titanium dioxide (TiO₂) nanoparticles magnified 100,000 times (500 nm×200 nm in size). In this case, the sample is coated with platinum (Pt) having a diameter of about 10 nm, and then measured using a field emission scanning electron microscope (FE-SEM).

In FIG. 3, an upper line represents IR data of an aldehyde silane solution, an intermediate line represents IR data of aldehyde group (—CHO) group-grafted titanium dioxide (TiO₂) nanoparticles, and a lower line represents IR data of hydroxyl (—OH) group-bound titanium dioxide (TiO₂) nanoparticles. In the intermediate line, a peak of 1723 cm⁻¹ represents an aldehyde carbonyl (C═O) group. The presence of a carbonyl (C═O) group peak (1723 cm⁻¹) indicates that an aldehyde (—CHO) group is engrafted into the titanium dioxide (TiO₂) nanoparticles.

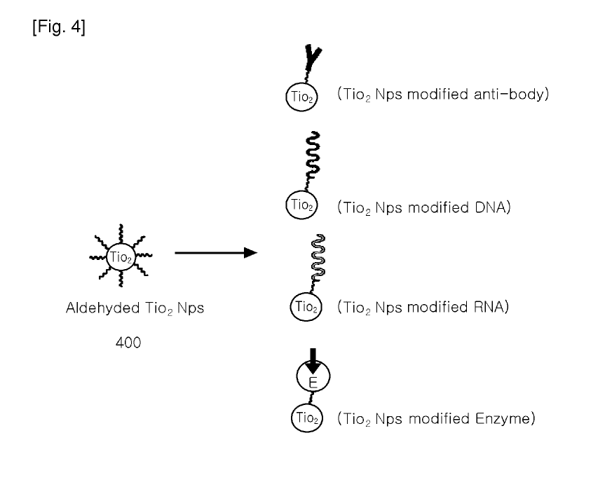

FIG. 4 is a diagram illustrating the titanium dioxide (TiO₂) nanoparticles immobilized by a bio-material according to one exemplary embodiment of the present invention.

Here, the bio-material used herein includes an antibody, DNA, RNA and an enzyme. More particularly, the bio-material includes antibody-immobilized titanium dioxide (TiO₂) nanoparticles 410, DNA-immobilized titanium dioxide (TiO₂) nanoparticles 420, RNA-immobilized titanium dioxide (TiO₂) nanoparticles 430, and enzyme-immobilized titanium dioxide (TiO₂) nanoparticles.

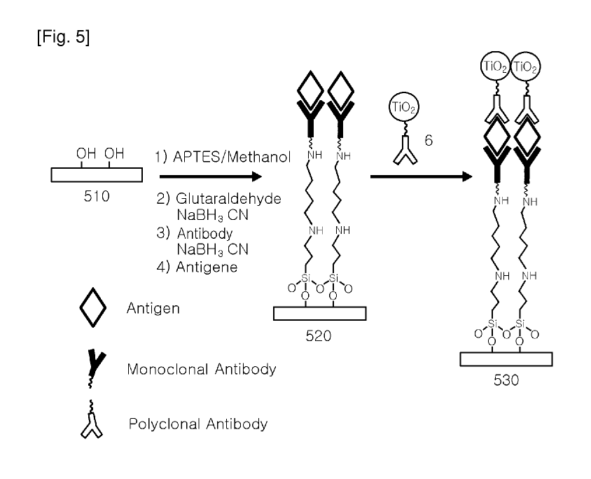

FIG. 5 is a schematic view illustrating an operation of measuring titanium dioxide (TiO₂) nanoparticles in a resonant reflection biosensor using antibody labeled with the titanium dioxide (TiO₂) nanoparticles according to the present invention.

To label antibody with the titanium dioxide (TiO₂) nanoparticles has an effect to improve sensitivity of a resonant reflection biosensor in the use of the labeled antibody in the resonant reflection biosensor. Here, a surface of Si₃N₄-coated resonant reflection filter is first treated with O₂ plasma to form a hydroxyl (—OH) group (510).

When the hydroxyl (—OH) group is formed in the surface of the resonant reflection filter, the resonant reflection filter is self-assembled using 3-aminopropyltriethoxysiliane (APTES), and an aldehyde (—CHO) group is engrafted into the self-assembled resonant reflection filter using glutaraldehyde. Then, antibody and antigen are engrafted into the aldehyde (—CHO) group-engrafted resonant reflection filter (520).

When the surface treatment of the resonant reflection filter is completed, the antibody labeled with the titanium dioxide (TiO₂) nanoparticles is attached to the surface-treated resonant reflection filter. In this case, the specific binding of the antibody and the antigen makes it possible to improve the sensitivity of the resonant reflection biosensor.

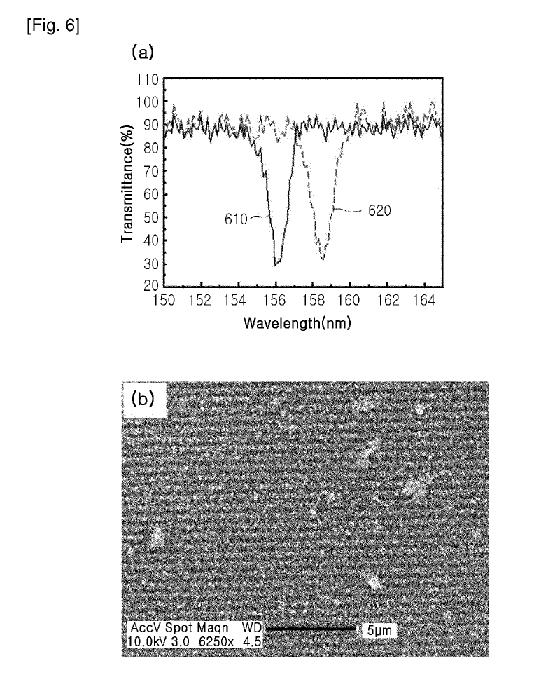

FIG. 6 is a diagram illustrating the spectrum results of the titanium dioxide (TiO₂) nanoparticles, which are immobilized by the bio-material, in a resonant reflection biosensor according to the present invention.

FIG. 6(a) shows the spectrum results determined through the resonant reflection biosensor. In the graph, the first peak 610 is obtained from the initial spectrum results, and the second peak 620 represents the spectrum results obtained when the antibody labeled with the titanium dioxide (TiO₂) nanoparticles according to the present invention is specifically bound to antigen. As seen in the graph, it is confirmed that the peak in the graph is shifted by 3.4 nm toward the right side.

FIG. 6(b) shows an FE-SEM photograph of the second peak 620. From the FE-SEM photograph, it is confirmed that many titanium dioxide (TiO₂) nanoparticles are attached to a substrate.

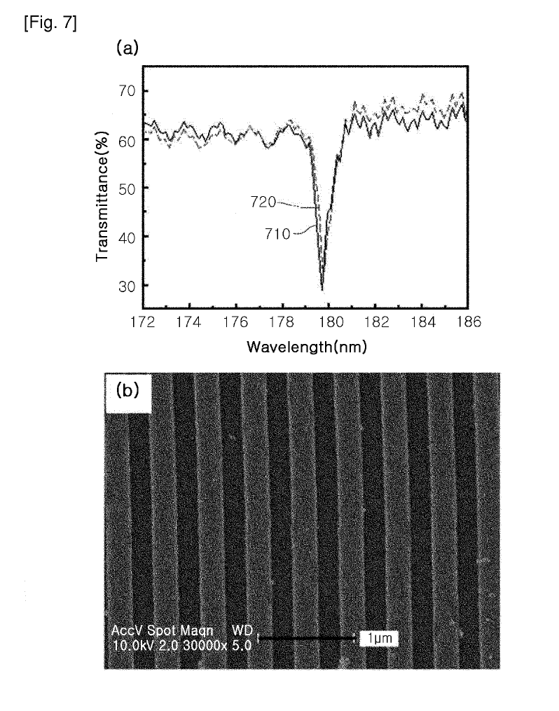

FIG. 7 is a diagram illustrating the spectrum results of the titanium dioxide (TiO₂) nanoparticles in the resonant reflection biosensor when a bio-material is not immobilized on the titanium dioxide (TiO₂) nanoparticles. In this case, FIG. 7 shows the spectrum results and the FE-SEM photograph of the

titanium dioxide (TiO₂) nanoparticles that are free from the specific interaction of antigen and antibody as shown in FIG. 6.

While the present invention has been shown and described in connection with the exemplary embodiments, it will be apparent to those skilled in the art that modifications and variations can be made without departing from the spirit and scope of the invention as defined by the appended claims.

1. A titanium dioxide (TiO₂) nanoparticle immobilized by a bio-material, comprising:
titanium dioxide (TiO₂) having a hydroxyl (—OH) group formed in a surface thereof;
an aldehyde (—CHO) group layer engrafted into the hydroxyl (—OH) group of titanium dioxide (TiO₂) using a self-assembly method; and
a bio-material immobilized on the aldehyde (—CHO) group layer.

2. The titanium dioxide (TiO₂) nanoparticle of claim 1, wherein the titanium dioxide (TiO₂) has the hydroxyl (—OH) group formed through the reaction with a piranha solution.

3. The titanium dioxide (TiO₂) nanoparticle of claim 1, wherein the aldehyde (—CHO) group layer is formed through the reaction of an aldehyde silane solution with the titanium dioxide (TiO₂) having a hydroxyl (—OH) group formed in the surface thereof.

4. The titanium dioxide (TiO₂) nanoparticle of claim 1, wherein the bio-material is selected from the group consisting of protein, DNA, RNA and enzyme.

5. A method for immobilizing a bio-material on titanium dioxide (TiO₂) nanoparticles, the method comprising:
binding hydroxyl (—OH) group to titanium dioxide (TiO₂) nanoparticles through the reaction with a piranha solution;
forming aldehyde (—CHO) group on the hydroxyl (—OH)-bound titanium dioxide (TiO₂) nanoparticles through the reaction with an aldehyde silane solution; and
immobilizing a bio-material on the titanium dioxide (TiO₂) nanoparticles through the reaction with the aldehyde group (—CHO) of the titanium dioxide (TiO₂).

6. The method of claim 5, wherein the binding of a hydroxyl (—OH) group comprises:
heating the titanium dioxide (TiO₂) nanoparticles in a piranha solution; and
separating the hydroxyl (—OH)-bound titanium dioxide (TiO₂) nanoparticles by using a centrifuge after the heating operation.

7. The method of claim 6, wherein the separating of the titanium dioxide (TiO₂) nanoparticles further comprises: washing the titanium dioxide (TiO₂) nanoparticles with double-distilled water.

8. The method of claim 5, wherein the forming of an aldehyde (—CHO) group comprises:
heating the hydroxyl (—OH)-bound titanium dioxide (TiO₂) nanoparticles in an aldehyde silane solution; and
separating the hydroxyl (—OH)-bound titanium dioxide (TiO₂) nanoparticles having aldehyde (—CHO) group formed in external surfaces thereof from the aldehyde silane solution by using a centrifuge.

9. The method of claim 8, wherein dimethyl sulfoxide (DMSO) is used to separate the titanium dioxide (TiO₂) nanoparticles having aldehyde (—CHO) group formed in external surfaces thereof.

10. The method of claim 5, wherein the immobilizing of a bio-material on the titanium dioxide (TiO₂) nanoparticles is carried out by immobilizing one of a protein, DNA, RNA and an enzyme on the titanium dioxide (TiO₂) nanoparticles having aldehyde (—CHO) group formed in external surfaces thereof.

11. A method for immobilizing a bio-material on titanium dioxide (TiO₂) nanoparticles, the method comprising:
binding hydroxyl (—OH) group to titanium dioxide (TiO₂) nanoparticles through the reaction with a piranha solution;
forming amino (—NH₂) group on the hydroxyl (—OH)-bound titanium dioxide (TiO₂) nanoparticles through the reaction with an aminosilane (3-aminopropyltriethoxysilane) solution;
forming aldehyde (—CHO) group on the amino group (—NH₂)-grafted titanium dioxide (TiO₂) nanoparticles through the reaction with glutaraldehyde; and
immobilizing a bio-material on the titanium dioxide (TiO₂) nanoparticles through the reaction with the aldehyde group (—CHO) of the titanium dioxide (TiO₂).

12. A method for immobilizing a bio-material on titanium dioxide (TiO₂) nanoparticles, the method comprising:
binding hydroxyl (—OH) group to titanium dioxide (TiO₂) nanoparticles through the reaction with a piranha solution;
forming amino (—NH₂) group on the hydroxyl (—OH)-bound titanium dioxide (TiO₂) nanoparticles through the reaction with an aminosilane (3-aminopropyltriethoxysilane) solution;
engrafting maleimido group into the amino group (—NH₂)-grafted titanium dioxide (TiO₂) nanoparticles using succinimidyl-4-(p-maleimide phenyl)butyrate (SMPB) as a cross-linker;
engrafting 3Cys-protein G into the maleimido group-grafted titanium dioxide (TiO₂) nanoparticles when the maleimido group is engrafted into the titanium dioxide (TiO₂) nanoparticles; and
immobilizing a bio-material on the titanium dioxide (TiO₂) nanoparticles through the reaction with the 3Cys-protein G of the titanium dioxide (TiO₂) nanoparticles.
